(19) United States
(12) Patent Application Publication
(10) Pub. No.: US 2023/0407335 A1
(43) Pub. Date: Dec. 21, 2023

(54) Methods for Preparing Genetically Modified Cells

(71) Applicants: Shanghai Cellular Biopharmaceutical Group Ltd., Shanghai (CN); CELLULAR BIOMEDICINE GROUP INC., Rockville, MD (US); CELLULAR BIOMEDICINE GROUP HK LIMITED, Hong Kong (CN)

(72) Inventors: Shichao QIN, Shanghai (CN); Lili ZHAI, Shanghai (CN); Zizhen GONG, Shanghai (CN); Fei WANG, Shanghai (CN); Dijun ZHAO, Shanghai (CN); Junfeng WU, Shanghai (CN); Li ZHANG, Shanghai (CN); Hanqing ZHANG, Shanghai (CN); Luyi ZHANG, Shanghai (CN)

(21) Appl. No.: 18/251,469

(22) PCT Filed: Nov. 4, 2021
(86) PCT No.: PCT/US21/72242

§ 371 Date: May 2, 2023

(30) Foreign Application Priority Data
Nov. 6, 2020 (CN) 2020112334494

(51) Int. Cl. C12N 15/86; C12N 5/0783; C07K 14/725; C07K 14/73; C07K 14/705

(52) U.S. Cl. CPC C12N 15/86; C12N 5/0636; C07K 14/7051; C07K 14/70514; C07K 14/70517; C12N 2501/515; C12N 2501/51; C12N 2501/505; C12N 2740/15043

(57) ABSTRACT

The present disclosure provides methods for improving the preparation efficiency of genetically modified immune cells and improving the quality of immune cells.

### CROSS REFERENCE TO RELATED APPLICATION

The present application claims priority to Chinese patent application No. 2020112334494, filed on Nov. 6, 2020, which is hereby incorporated by reference in its entirety.

### TECHNICAL FIELD

The present invention relates to the field of biotechnology, and specifically to methods for preparing genetically modified cells.

### BACKGROUND

In recent years, considerable progress has been made in cancer immunotherapy. In cellular immunotherapy, also known as adoptive cell therapy, a patient's immune cells are collected for genetic modification or selective expansion to enhance the antigen-specific immune responses. However, the methods of preparing immune cells are relatively complicated. Insufficiency of any components of the process, such as process flow, equipment and facilities, and reagents, can have an important impact on the quality of the final cell preparation, which in turn affects clinical effects.

Traditional immune cell preparation methods have disadvantages such as complex procedures and long duration. The relatively long time period not only is likely to cause excessive differentiation and senescence of the cells during the culture process, but also can indirectly affect the disease progression of the patients. All of these can increase costs, reduce clinical efficacy, and create difficulty to meet the clinical needs of cellular immunotherapy.

Therefore, there is a need to develop more robust and efficient methods of generating immune cells for clinical use.

### SUMMARY

The present disclosure provides a method for preparing genetically modified immune cells. The method may comprise the steps of: (a) providing a sample containing immune cells (to be genetically modified); (b) sorting the sample to obtain a first immune cell population enriched in immune cells; (c) activating the first immune cell population to obtain a second immune cell population; (d) culturing the second immune cell population (e.g., pre-transduction or pre-transfection culturing, also called pre-culturing) to obtain a third immune cell population; (e) genetically modifying (e.g., transducing (for example with viral vectors), or transfecting) the third immune cell population to obtain a fourth immune cell population; and (f) culturing the fourth immune cell population (e.g., post-transduction or post-transfection culturing, also called post-culturing) to obtain genetically modified immune cells.

In step (c), activating beads (e.g., beads coated with activating agents) may be used for activating the first immune cell population. For example, the activating beads may be activating magnetic beads.

The activating beads may be microbeads which have a diameter (or mean diameter) ranging from about 1 μm to about 10 μm, from about 2 μm to about 8 μm, or from about 4 μm to about 5 μm.

In step (c), the activating may be performed with a bead-to-cell ratio (number ratio) ranging from about 0.1 to about 10 (from about 0.1:1 to about 10:1), from about 0.2 to about 8 (from about 0.2:1 to about 8:1), from about 0.5 to about 8 (from about 0.5:1 to about 8:1), from about 0.1 to about 8 (from about 0.1:1 to about 8:1), from about 0.5 to about 5 (from about 0.5:1 to about 5:1), from about 0.5 to about 4 (from about 0.5:1 to about 4:1), from about 0.5 to about 3 (from about 0.5:1 to about 3:1), from about 0.5 to about 2 (from about 0.5:1 to about 2:1), from about 0.5 to about 1 (from about 0.5:1 to about 1:1), from about 1 to about 8 (from about 1:1 to about 8:1), from about 1 to about 6 (from about 1:1 to about 6:1), from about 1 to about 5 (from about 1:1 to about 5:1), from about 1 to about 3 (from about 1:1 to about 3:1), from about 0.5 to about 5 (from about 0.5:1 to about 5:1), from about 1 to about 2 (from about 1:1 to about 2:1), about 0.1:1, about 0.2:1, about 0.5:1, about 0.8:1, about 1:1, about 1.2:1, about 1.5:1, about 1.8:1, about 2:1, about 2.5:1, about 3:1, about 3.5:1, about 4:1, about 4.5:1, or about 5:1. The ratio of the number of beads to the number of cells may range from about 0.5:1 to about 5:1 (about 0.5-5:1), or from about 1:1 to about 5:1 (about 1-5:1).

In certain embodiments, in step (c), the density of the first immune cell population may range from about 0.5×10⁶ to about 10×10⁶ cells/ml.

In one embodiment, step (c) comprises: mixing the first immune cell population and activating beads to form a mixture, and incubating the mixture for a period of time t_{c}, to obtain a second immune cell population. In one embodiment, t_{c} may range from about 12 hours to about 24 hours.

In step (c), the activating or incubating may be performed for about 2 hours to about 1 week, about 2 hours to about 6 days, about 2 hours to about 5 days, about 2 hours to about 4 days, about 2 hours to about 3 days, about 2 hours to about 2 days, about 2 hours to about 1 day, about 2 hours to about 20 hours, about 2 hours to about 16 hours, about 4 hours to about 5 days, about 4 hours to about 96 hours, about 4 hours to about 48 hours, about 4 hours to about 36 hours, about 4 hours to about 24 hours, about 4 hours to about 20 hours, about 4 hours to about 16 hours, about 16 hours to about 48 hours, about 16 hours to about 40 hours, about 16 hours to about 36 hours, about 16 hours to about 24 hours, about 2 hours, about 4 hours, about 5 hours, about 6 hours, about 8 hours, about 10 hours, about 12 hours, about 18 hours, about 20 hours, about 24 hours, about 30 hours, about 36 hours, about 40 hours, about 48 hours, about 50 hours, about 55 hours, about 60 hours, about 65 hours, about 72 hours, about 84 hours, about 96 hours, about 4.5 days, about 5 days, about 5.5 days, about 6 days, about 6.5 days, or about 1 week.

In step (e), the genetically modifying may be transducing or transfecting.

In step (e), the genetically modifying may comprise introducing into the third immune cell population a polynucleotide encoding a chimeric antigen receptor (CAR) or a T cell receptor (TCR).

In step (e), the genetically modifying may comprise transducing the third immune cell population with lentiviral vectors, gamma-retroviral vectors, alpha-retroviral vectors, or adenoviral vectors. In certain embodiments, the genetically modifying may comprise transducing the third immune cell population with lentiviral vectors.

In step (e), the viral vectors and the third immune cell population may be mixed and incubated for a period of time tₑ. Centrifugation may or may not be carried out after the incubation.

In step (f), during culturing, perfusion may be used based on the density of the immune cells in a culture system in which the immune cells are cultured. For example, when the density of the immune cells is less than 2×10⁶ cells/ml, no perfusion is carried out. When the density of the immune cells is greater than or equal to 2×10⁶ cells/ml and less than 4×10⁶ cells/ml, perfusion may be carried out at a rate of 0.5 V/day (volume per day) to 1 V/day, 0.6 V/day to 1 V/day, 0.7 V/day to 1 V/day, 0.8 V/day to 1 V/day, 0.5 V/day to 0.7 V/day, or 0.5 V/day to 0.8 V/day, where V is the volume of the culture system. When the density of the immune cells is greater than or equal to 4×10⁶ cells/ml, perfusion may be carried out at a rate of 1 V/day to 2 V/day, 1 V/day to 1.3 V/day, 1 V/day to 1.5 V/day, 1 V/day to 1.8 V/day, 1.3 V/day to 2 V/day, 1.5 V/day to 2 V/day, or 1.8 V/day to 2 V/day, where V is the volume of the culture system.

In one embodiment, step (b) comprises: mixing the sample with sorting beads (e.g., sorting magnetic beads) to form a mixture, incubating the mixture for a period of time t_{b}, and then selecting a first immune cell population enriched in immune cells. In certain embodiments, t_{b} may range from about 10 minutes to about 30 minutes, or from about 10 minutes to about 25 minutes.

In step (b), the sorting or incubating time may range from about 10 minutes to about 5 hours, about 10 minutes to about 4 hours, about 10 minutes to about 3 hours, about 10 minutes to about 2 hours, about 1 minute to about 60 minutes, about 2 minutes to about 45 minutes, for about 5 minutes to about 30 minutes, about 5 minutes to about 20 minutes, about 5 minutes to about 15 minutes, about 10 minutes to about 60 minutes, about 10 minute to about 50 minutes, about 10 minute to about 45 minutes, about 2 minutes to about 45 minutes, about 2 minutes to about 30 minutes, about 2 minutes to about 20 minutes, about 2 minutes to about 15 minutes, about 2 minutes to about 10 minutes, about 5 minutes to about 45 minutes, about 5 minutes to about 40 minutes, about 5 minutes to about 10 minutes, about 5 minutes, about 6 minutes, about 7 minutes, about 8 minutes, about 9 minutes, about 10 minutes, about 15 minutes, about 20 minutes, about 25 minutes, or about 30 minutes.

The total time t_{(b-f)} from steps (b) to (f) may range from about 4 days to about 5 days. Step (b), step (c), step (d), step (e) and step (f) of the method, or all steps of the method, may be performed in about 2 days to about 5 days, about 3 days to about 4 days, about 2 days to about 10 days, about 2 days to about 9 days, about 2 days to about 8 days, about 2 days to about 7 days, about 2 days to about 6 days, about 2 days to about 5 days, about 2 days to about 4 days, about 3 days to about 10 days, about 3 days to about 9 days, about 3 days to about 8 days, about 3 days to about 7 days, about 3 days to about 6 days, about 3 days to about 5 days, about 2 days, about 3 days, about 4 days, about 5 days, about 6 days, about 7 days, about 8 days, about 9 days, or about 10 days.

In step (d), the pre-culturing time t_{d} may range from about 1.5 days to about 3 days, or from about 1.5 days to about 2.5 days. The time for performing step (d), or the culture time t_{d} of step (d), may range from about 0.5 days to about 10 days, about 2 days to about 5 days, about 3 days to about 4 days, about 2 days to about 10 days, about 2 days to about 9 days, about 2 days to about 8 days, about 2 days to about 7 days, about 2 days to about 6 days, about 2 days to about 5 days, about 2 days to about 4 days, about 3 days to about 10 days, about 3 days to about 9 days, about 3 days to about 8 days, about 3 days to about 7 days, about 3 days to about 6 days, about 3 days to about 5 days, about 0.5 days, about 1 days, about 1.5 days, about 2 days, about 2.5 days, about 3 days, about 3.5 days, about 4 days, about 4.5 days, about 5 days, about 6 days, about 7 days, about 8 days, about 9 days, or about 10 days.

In step (e), the incubation time tₑ (when the cells are incubated with the vectors such as viral vectors or non-viral vectors) may range from about 0.5 days to about 2.5 days, or from about 1 day to about 2 days. The time for performing step (e), or the incubation time tₑ (when the cells are incubated with the vectors such as viral vectors or non-viral vectors) of step (e), may range from about 0.5 days to about 10 days, about 2 days to about 5 days, about 3 days to about 4 days, about 2 days to about 10 days, about 2 days to about 9 days, about 2 days to about 8 days, about 2 days to about 7 days, about 2 days to about 6 days, about 2 days to about 5 days, about 2 days to about 4 days, about 3 days to about 10 days, about 3 days to about 9 days, about 3 days to about 8 days, about 3 days to about 7 days, about 3 days to about 6 days, about 3 days to about 5 days, about 0.5 days, about 1 days, about 1.5 days, about 2 days, about 2.5 days, about 3 days, about 3.5 days, about 4 days, about 4.5 days, about 5 days, about 6 days, about 7 days, about 8 days, about 9 days, or about 10 days.

In step (f), the culturing time t_{f} after transfection/transduction may range from about 1 day to about 3.5 days, or from about 1.5 days to about 3 days. The culture time t_{f} of step (f) may range from about 1 day to about 3.5 days. The time for performing step (f), or the culture time t_{f} of step (f), may range from about 0.5 days to about 10 days, about 2 days to about 5 days, about 3 days to about 4 days, about 2 days to about 10 days, about 2 days to about 9 days, about 2 days to about 8 days, about 2 days to about 7 days, about 2 days to about 6 days, about 2 days to about 5 days, about 2 days to about 4 days, about 3 days to about 10 days, about 3 days to about 9 days, about 3 days to about 8 days, about 3 days to about 7 days, about 3 days to about 6 days, about 3 days to about 5 days, about 0.5 days, about 1 days, about 1.5 days, about 2 days, about 2.5 days, about 3 days, about 3.5 days, about 4 days, about 4.5 days, about 5 days, about 6 days, about 7 days, about 8 days, about 9 days, or about 10 days.

Step (f) of the method may further comprise: harvesting the cultured fourth immune cell population, when the cell number or cell density of the cultured fourth immune cell population reaches a predetermined value. In certain embodiments, the predetermined value ranges from about 2×10⁶ cells/ml to about 20×10⁶ cells/ml.

In certain embodiments, in step (e), the viruses and the third immune cell population may be mixed (or the viruses may be added to the third immune cell population), and incubated for a period of time tₑ to obtain an incubation mixture. The incubation mixture may be diluted 0.5-2 folds, or 0.75-1.5 folds (by volume) with a culture medium so as to obtain a diluted incubation mixture.

In one embodiment, in step (e), the (diluted) incubation mixture is incubated for about 0.5 days to about 1.5 days, then inoculated into the cell culture medium (e.g., in a bioreactor, such as Xuri Wave), and is cultured for about 1 day to about 7 days.

In step (e), the time for genetically modifying (e.g., transducing or transfecting), incubating or culturing may range from about 0.5 days to about 10 days, about 2 days to about 5 days, about 3 days to about 4 days, about 2 days to about 10 days, about 2 days to about 9 days, about 2 days to about 8 days, about 2 days to about 7 days, about 2 days to about 6 days, about 2 days to about 5 days, about 2 days to about 4 days, about 3 days to about 10 days, about 3 days to about 9 days, about 3 days to about 8 days, about 3 days to about 7 days, about 3 days to about 6 days, about 3 days to about 5 days, about 0.5 days, about 1 days, about 1.5 days, about 2 days, about 2.5 days, about 3 days, about 3.5 days, about 4 days, about 4.5 days, about 5 days, about 6 days, about 7 days, about 8 days, about 9 days, or about 10 days.

The sample may be blood, cells, fresh apheresis, cryopreserved apheresis, PBMC collections, or combinations thereof. The sample may be peripheral blood, immune cells, monocyte collections, or peripheral blood mononuclear cells (PBMCs), e.g., from a subject (e.g., patient), or a plurality of subjects.

The immune cells may be T cells, T cell subsets (e.g., Tnaive or naive T cell; Tcm or central memory T cell, etc.), natural killer (NK) cells, or a combination thereof.

In certain embodiments, the sample is washed before being sorted. The washing may comprise the steps of: adding a washing liquid to the sample, mixing, centrifuging, and removing a supernatant to obtain a precipitate. In one embodiment, washing is performed on equipment selected from Sepax 2, Sepax C-pro, Sefia, Lovo, CS 5+, CS Elite or Prodigy. In another embodiment, the washing is carried out on Sepax C-pro equipment. In yet another embodiment, a “BeadWash” program of Sepax C-pro is used for sample washing and magnetic bead incubation, and BeadWash parameters comprise one or more of the following:

| Parameter | Set value | |
|---|---|---|
| Initial Volume (volume of the sample) | 10-440 | ml |
| Dilution ratio (volume ratio of washing liquid to | 1-2 | |
| liquid to be washed) | | |
| Dilution speed (speed of adding washing liquid) | 17-60 | ml/min |
| Intermediate volume (volume of the precipitate after | 5 to 20 ml | |
| centrifugation) | | |
| Pre-wash cycle (number of washes before the | 1-3 | |
| incubation step) | | |
| Pre-wash force (centrifugal force in washing step) | 300 to 600 g | |
| Pre-wash time (centrifugation time in washing step) | 300 to 600 s | |
| Reagent volume | 10-30 | ml |
| Incubation volume | Calculated | |
| | incubation volume | |
| | of magnetic beads | |
| Incubation time (time for incubation) | 10-30 | min |
| Post wash cycle (number of washes after | 1-3 | |
| incubation) | | |
| Post wash force (centrifugal force in washing step | 300-600 | g |
| after incubation) | | |
| Post wash time (centrifugation time in washing step | 300-900 | s |
| after incubation) | | |
| Final volume (volume of adjusted system after | 10-100 | ml |
| incubation) | | |

The washing may comprise using a human serum albumin (HSA) solution having a HSA final concentration of about 0.1% to about 30%, about 0.1% to about 10%, about 0.1% to about 25%, about 0.1% to about 20%, about 0.1% to about 15%, about 0.1% to about 8%, about 0.1% to about 6%, or about 0.1% to about 5%.

The washing may comprise centrifuging the sample using a centrifugal force ranging from about 100×g to about 1,000×g, from about 200×g to about 400×g, from about 100×g to about 800×g, from about 100×g to about 600×g, from about 100×g to about 500×g, from about 200×g to about 800×g, from about 200×g to about 600×g, or from about 200×g to about 500×g.

The washing may comprise centrifuging the sample for about 100 seconds to about 600 seconds, about 300 seconds to about 400 seconds, about 50 seconds to about 1,000 seconds, about 100 seconds to about 800 seconds, about 100 seconds to about 500 seconds, about 200 seconds to about 800 seconds, about 200 seconds to about 600 seconds, about 200 seconds to about 500 seconds, about 300 seconds to about 800 seconds, or about 300 seconds to about 500 seconds.

The washing may comprise diluting the sample about 0 (no dilution) to about 5 folds, about 1 fold to about 5 folds, about 1 fold to about 4 folds, about 1 fold to about 3 folds, or about 2 folds to about 3 folds.

The washing may comprise performing/repeating the washing cycle for 1 to 5 times, 1 to 4 times, 1 to 3 times, 1 to 2 times, 2 to 5 times, 2 to 4 times, or 2 to 3 times.

The washing step may have an output volume ranging from about 5 ml to about 400 ml, from about 20 ml to about 100 ml, from about 10 ml to about 300 ml, from about 10 ml to about 200 ml, from about 10 ml to about 100 ml, from about 20 ml to about 400 ml, from about 20 ml to about 300 ml, from about 20 ml to about 200 ml, from about 50 ml to about 400 ml, from about 50 ml to about 300 ml, from about 50 ml to about 200 ml, or from about 50 ml to about 100 ml.

In step (b), the sorting may comprise positive sorting and/or negative sorting.

In certain embodiments, the marker for positive sorting may be CD4, CD8, CD62L, CD3, CD56, or a combination thereof.

In certain embodiments, the marker for negative sorting may be CD14, CD19, CD269, or a combination thereof.

In step (b), the sorting may comprise using anti-CD4 and/or anti-CD8 antibodies or fragments thereof. In one embodiment, in step (b), the sorting comprises sorting by means of adding sorting magnetic beads which contain binding agents specific to at least one immune cell surface marker (such as CD4 and/or CD8). The sorting magnetic beads bind to the immune cell surface marker(s) through the binding agents to form a sorting magnetic bead-cell complex, so as to obtain a first immune cell population enriched in immune cells.

In one embodiment, the binding agents are antibodies or fragments thereof. In one embodiment, the antibodies are specific antibodies. In one embodiment, the antibodies are anti-CD4 antibodies, anti-CD8 antibodies, or a combination thereof.

In one embodiment, the cell surface markers are: CD4, CD8, or a combination thereof.

In one embodiment, the binding agents specifically bind to the cell surface marker(s).

In one embodiment, the sorting magnetic beads are sorting magnetic beads containing anti-CD4 antibodies and/or anti-CD8 antibodies.

In one embodiment, the washing liquid is a buffer.

In one embodiment, the sorting liquid is a buffer containing sorting magnetic beads.

In one embodiment, the buffer is a pH 6.8-7.4 phosphate-buffered saline (PBS) buffer.

In one embodiment, in step (c), the activating magnetic beads contain anti-CD3 antibodies, anti-CD28 antibodies, or a combination thereof.

In one embodiment, the activating magnetic beads are Dynabeads®.

In certain embodiments, in step (c), for the activating step, the density of the immune cells (e.g., the first immune cell population, or the second immune cell population) may range from about 0.5×10⁶ cells/ml to about 10×10⁶ cells/ml. In step (c), the activating may be performed with an immune cell density (e.g., density of the first immune cell population, or the second immune cell population) ranging from about 0.5×10⁶ cells/ml to about 10×10⁶ cells/ml, from about 2×10⁶ cells/ml to about 3×10⁶ cells/ml, from about 0.5×10⁶ cells/ml to about 8×10⁶ cells/ml, from about 0.5×10⁶ cells/ml to about 5×10⁶ cells/ml, from about 1×10⁶ cells/ml to about 8×10⁶ cells/ml, from about 1×10⁶ cells/ml to about 6×10⁶ cells/ml, from about 1×10⁶ cells/ml to about 5×10⁶ cells/ml, from about 1×10⁶ cells/ml to about 4×10⁶ cells/ml, from about 1×10⁶ cells/ml to about 3×10⁶ cells/ml, about 0.5×10⁶ cells/ml, about 1×10⁶ cells/ml, about 1.5×10⁶ cells/ml, about 2×10⁶ cells/ml, about 2.5×10⁶ cells/ml, about 3×10⁶ cells/ml, about 3.5×10⁶ cells/ml, about 4×10⁶ cells/ml, about 4.5×10⁶ cells/ml, about 5×10⁶ cells/ml, about 5.5×10⁶ cells/ml, about 6×10⁶ cells/ml, about 6.5×10⁶ cells/ml, about 7×10⁶ cells/ml, about 7.5×10⁶ cells/ml, about 8×10⁶ cells/ml, about 8.5×10⁶ cells/ml, about 9×10⁶ cells/ml, about 9.5×10⁶ cells/ml, or about 10×10⁶ cells/ml.

In certain embodiments, in step (e), the transduction/transfection may be non-viral transfection (including an electroporation system, such as a Neon transfection system and a MaxCyte transfection system), and viral transduction (such as using a lentivirus system, an adenovirus system and an adeno-associated virus vector).

In one embodiment, in step (e), in the transduction/transfection step, the ratio of the number of viruses to the number of cells ranges from about 1:1 to about 10:1. In another embodiment, in step (e), viral vectors are used for the transduction, and the multiplicity of infection or MOI is about 0 to about 1,000, or about 1 to about 10.

In one embodiment, in step (e), the viruses (viral vectors) are lentiviruses (lentiviral vectors).

In one embodiment, the gene is a tumor-killing gene.

In one embodiment, in step (f), the culturing is carried out in Wave equipment.

In one embodiment, the culturing is carried out in a wave culture bag.

In one embodiment, the wave culture bag has a capacity of 2 L-10 L.

In one embodiment, Wave parameters are as follows: temperature of 35-39° C., gas flow of 0.08-0.15 L/min, 4-6% of CO₂, rocking speed of 10-18 rpm, and rocking angle of 6-10°.

In one embodiment, in step (f), after culturing, the activating beads are removed using a magnetic device (e.g., CTS™ DynaMag™ Magnet).

In one embodiment, step (f) further comprises concentrating the cultured fourth immune cell population. The concentrating may be carried out on the Sepax C-pro equipment.

In one embodiment, a “CultureWash” program of Sepax C-pro is used for concentration. The “CultureWash” parameters may comprise one or more of the following:

| Parameter | Set value |
|---|---|
| Initial volume (volume of the cell mixture | 10 to 1200 ml |
| after removing the activating beads) | |
| Final Volume (concentrated volume) | 5-500 ml |
| Intermediate Volume (volume of the | 5 to 50 ml |
| precipitate after centrifugation) | |
| G-force (centrifugal force in washing and | 300 to 400 g |
| concentration steps) | |
| Sedimentation time (centrifugal force in | 300 to 600 s |
| washing step) | |
| Wash Cycles (number of washes) | 1 to 3 |

The present disclosure provides genetically modified immune cells prepared by the present method.

The present disclosure also provides a cell preparation comprising the genetically modified immune cells.

Also encompassed by the present disclosure is a pharmaceutical composition comprising the genetically modified immune cells. The pharmaceutical composition may further comprise a pharmaceutically acceptable carrier.

It should be understood that the above-mentioned technical features of the present invention and the technical features specifically described in the following (such as the embodiments) can be combined with each other, so as to form new or preferred technical solutions.

### DETAILED DESCRIPTION

The present disclosure provides methods for improving the preparation efficiency of genetically modified immune cells and improving the quality of immune cells.

The present method can quickly prepare genetically modified immune cells that are of high quality, which ensure clinical efficacy. The present methods can lead to high transduction efficiency of the manufactured T cells and a high transgene expression by the genetically modified immune cells.

The clinical manufacture of genetically modified T cells is currently a complex process that generally starts with obtaining the patient's peripheral blood mononuclear cells (PBMCs). Current protocols feature a leukapheresis step. PBMCs are often enriched for T cells and activated prior to genetic modification with viral or nonviral vectors. The modified T cells are then expanded in order to reach the cell numbers required for treatment, after which the cells are finally formulated and/or cryopreserved prior to reinfusion into the patient.

The present disclosure provides a method for preparing genetically modified immune cells. The method may comprise the steps of: (a) providing a sample containing immune cells (to be genetically modified); (b) sorting the sample to obtain a first immune cell population enriched in immune cells; (c) activating the first immune cell population to obtain a second immune cell population; (d) culturing the second immune cell population (e.g., pre-transduction or pre-transfection culturing, also called pre-culturing) to obtain a third immune cell population; (e) genetically modifying (e.g., transducing (for example with viral vectors), or transfecting) the third immune cell population to obtain a fourth immune cell population; and (f) culturing the fourth immune cell population (e.g., post-transduction or post-transfection culturing, also called post-culturing) to obtain genetically modified immune cells.

In step (b), for the sorting step, the immune cells may be enriched through magnetic separation using antigen-binding molecules (e.g., antibodies or fragments thereof) specific for one or more cell surface markers on the surface of the immune cells, such as markers CD2, CD3, CD4, CD8, CD25, CD28, CD27, CD45RA, CD45RO, CD62L, CD95, CD127, CD137, alpha/beta TCR, gamma/delta TCR, CCR7, PD-1 and Lag3.

In step (c), microbeads (e.g., activating magnetic beads) may be used for activation.

The ratio of the number of activating beads to the number of cells may range from about 0.5:1 to about 5:1 (0.5-5:1), or from about 1:1 to about 5:1 (1-5:1). In certain embodiments, the ratio of the number of activating magnetic beads to the number of cells may range from about 0.1:1 to about 10:1, from about 0.2:1 to about 9:1, from about 0.3:1 to about 8:1, from about 0.4:1 to about 7:1, from about 0.5:1 to about 6:1, from about 0.6:1 to about 5:1, about 0.1:1, about 0.2:1, about 0.3:1, about 0.4:1, about 0.5:1, about 0.6:1, about 0.7:1, about 0.8:1, about 0.9:1, about 1:1, about 1.2:1, about 1.5:1, about 1.8:1, about 2:1, about 2.5:1, about 3:1, about 3.5:1, about 4:1, about 4.5:1, about 5:1, about 5.5:1, about 6:1, about 6.5:1, about 7:1, or about 8:1.

In certain embodiments, the microbeads are polymer microbeads. In certain embodiments, the microbeads are magnetic microbeads. In certain embodiments, the microbeads are magnetic polymer microbeads. In certain embodiments, the microbeads are superparamagnetic polymer microbeads. Polymers may include polystyrene, polyesters, polyethers, polyacrylates, polyacrylamides, polyamines, polyethylene imines, polyquarternium polymers, polyphosphazenes, polyvinylalcohols, polyvinylacetates, polyvinylpyrrolidones, block copolymers, or polyurethanes.

The microbeads may have a diameter (or a median or mean diameter) ranging from about 1 μm to about 50 μm, from about 1 μm to about 40 μm, from about 1 μm to about 30 μm, from about 1 μm to about 20 μm, from about 1 μm to about 15 μm, from about 1 μm to about 12 μm, from about 1 μm to about 10 μm, from about 1 μm to about 8 μm, from about 1 μm to about 6 μm, from about 1 μm to about 5 μm, from about 2 μm to about 10 μm, from about 2 μm to about 8 μm, from about 2 μm to about 6 μm, from about 3 μm to about 10 μm, from about 3 μm to about 8 μm, from about 3 μm to about 6 μm, from about 3 μm to about 5 μm, from about 4 μm to about 5 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, or about 4.5 μm.

In step (c), the cells may be activated with microbeads coated with activating agents to obtain activated immune cells (e.g., a second immune cell population).

The activating agents may be agonistic antibodies, cytokines, recombinant costimulatory molecules, small drug inhibitors, or combinations thereof. In certain embodiments, the activating agents are anti-CD3 and/or anti-CD28 antibodies or fragments thereof, coupled to microbeads, microparticles, microsphere or microstructures. In certain embodiments, the activating agents are microbeads coated with anti-CD3 and/or anti-CD28 antibodies or fragments thereof.

Activation of the cells may be performed by using cell densities between 0.2×10⁶ cells per ml to 4×10⁶ cells per ml, between 0.5×10⁶ cells per ml to 2×10⁶ cells per ml, or about 1×10⁶ cells per ml. Alternatively, the activation may be performed by using high cell densities between 4×10⁶ cells per ml to 2×10⁷ cells per ml, or between 4×10⁶ cells per ml to 1×10⁷ cells per ml.

The immune cell may be a T cell, a natural killer (NK) cell, a natural killer T cell, a lymphoid progenitor cell, a hematopoietic stem cell, a stem cell, a macrophage, or a dendritic cell.

In certain embodiments, the immune cells are derived from humans or non-human mammals (such as mice).

In certain embodiments, the cells include T cells and/or NK cells.

The genetic modification of immune cells may be performed by transduction, transfection or electroporation.

Transduction may be performed with lentiviruses, gamma-retroviruses, alpha-retroviruses or adenoviruses. Electroporation or transfection of the cells may be performed by introducing into the cells nucleic acids (DNA, mRNA, miRNA, antagomirs, ODNs), proteins, through site-specific nucleases (zinc finger nucleases, TALENs, CRISP/R), self-replicating RNA viruses (e.g. equine encephalopathy virus) or integration-deficient lentiviral vectors.

In one embodiment, the genetic modification of immune cells may be by transducing the cells with lentiviral vectors.

Viral transduction of the immune cells can be enhanced by the use of transduction enhancer reagents, especially transduction enhancer reagents including, but not limited to, polycationic reagents (polybrene, protamine sulphate, poly-L-lysine, peptides with a net positive charge), poloxamers, adhesion molecules such as fibronectin or modified fibronectin (RetroNectin), or protein targeting domains such as antibodies, antibody complexes, magnetic particles. The transduction enhancers can be provided in solution, coated on the cultivation chamber or coated on a carrier substance present in suspension/solution.

The genetically modified immune cells may be genetically modified to express a chimeric antigen receptor (CAR), a T cell receptor (TCR), or any accessory molecule, on their cell surface.

In certain embodiments, the genetically modified immune cell includes a T cell or an NK cell, such as, (i) a chimeric antigen receptor T cell (CAR-T cell); or (ii) a chimeric antigen receptor NK cell (CAR-NK cell).

In step (e), the viral vectors and the third immune cell population may be mixed and incubated for a period of time tₑ. Centrifugation may or may not be carried out after the incubation.

In step (f), during culturing, perfusion may be used based on the density of the immune cells in a culture system. For example, when the density of the immune cells is less than 2×10⁶ cells/ml, no perfusion is carried out. When the density of the immune cells is greater than or equal to 2×10⁶ cells/ml and less than 4×10⁶ cells/ml, perfusion may be carried out at a rate of 0.5 V/day (volume per day) to 1 V/day, where V is the volume of the culture system. When the density of the immune cells is greater than or equal to 4×10⁶ cells/ml, perfusion may be carried out at a rate of 1 V/day to 2 V/day, where V is the volume of the culture system.

In certain embodiments, in step (c), the density of the first immune cell population may range from about 0.5×10⁶ to about 10×10⁶ cells/ml.

The present method can prepare immune cells within a relatively short period of time.

The total time from steps (b) to (f), t_{(b-f)}, may range from about 4 days to about 5 days.

In one embodiment, step (b) comprises: mixing the sample with sorting magnetic beads to form a mixture, incubating the mixture for a period of time t_{b}, and then selecting the first immune cell population enriched in immune cells. In certain embodiments, t_{b} may range from about 10 minutes to about 30 minutes, or from about 10 minutes to about 25 minutes.

In one embodiment, step (c) comprises: mixing the first immune cell population and activating magnetic beads to form a mixture, and incubating the mixture for a period of time t_{c}, so as to obtain a second immune cell population. In one embodiment, t_{c} may range from about 12 hours to about 24 hours.

In step (d), the pre-culturing time t_{d} may range from about 1.5 days to about 3 days, or from about 1.5 days to about 2.5 days.

In step (e), the incubation time tₑ may range from about 0.5 days to about 2.5 days, or from about 1 day to about 2 days.

In step (f), the culturing time t_{f} after transfection/transduction may range from about 1 day to about 3.5 days, or from about 1.5 days to about 3 days.

Step (f) of the method may further comprise: harvesting the cultured fourth immune cell population, when the cell number or cell density of the cultured fourth immune cell population reaches a predetermined value. In certain embodiments, the predetermined value ranges from about 2×10⁶ cells/ml to about 20×10⁶ cells/ml.

In certain embodiments, in step (e), the viruses and the third immune cell population may be mixed, and incubated for a period of time tₑ to obtain an incubation mixture. The incubation mixture may be diluted 0.5-2 folds, or 0.75-1.5 folds, by volume, with a culture medium so as to obtain a diluted incubation mixture.

In one embodiment, in step (e), the (diluted) incubation mixture is incubated for about 0.5 to about 1.5 days, then inoculated into a cell culture medium (e.g., in a bioreactor such as Xuri Wave), and is cultured for about 1 day to about 7 days.

In the present method, the samples are not particularly limited. The sample may be blood, cells, fresh apheresis, cryopreserved apheresis, PBMC collections, or combinations thereof.

The immune cells may be T cells, natural killer (NK) cells, or a combination thereof.

In certain embodiments, the sample is washed before being sorted. The washing may comprise the steps of: adding a washing liquid to the sample, mixing, centrifuging, and removing a supernatant to obtain a precipitate. In one embodiment, washing is carried out on Sepax 2, Sepax C-pro, Sefia, Lovo, CS 5+, CS Elite or Prodigy equipment. In another embodiment, the washing is carried out on Sepax C-Pro equipment. Sepax C-Pro is a fully automated and closed cell processing system. It can be used to process cells to generate cell therapy products. It can be used in conjunction with software and kits to achieve a multifunctional combination of multiple processing steps, including but not limited to, enriching, separating, washing, concentrating, diluting and bagging cells from various sources (e.g., umbilical cord blood, bone marrow, peripheral blood, fat, cultured cells, etc.).

In yet another embodiment, a “BeadWash” program of Sepax C-pro is used for sample washing and magnetic bead incubation, and Beadwash parameters comprise one or more of the following:

| Parameter | Set value | |
|---|---|---|
| Initial Volume (volume of the sample) | 10-440 | ml |
| Dilution ratio (volume ratio of washing liquid to | 1-2 | |
| liquid to be washed) | | |
| Dilution speed (speed of adding washing liquid) | 17-60 | ml/min |
| Intermediate volume (volume of the precipitate after | 5 to 20 ml | |
| centrifugation) | | |
| Pre-wash cycle (number of washes before the | 1-3 | |
| incubation step) | | |
| Pre-wash force (centrifugal force in washing step) | 300 to 600 g | |
| Pre-wash time (centrifugation time in washing step) | 300 to 600 s | |
| Reagent volume | 10-30 | ml |
| Incubation volume | Calculated | |
| | incubation volume | |
| | of magnetic beads | |
| Incubation time (time for incubation) | 10-30 | min |
| Post wash cycle (number of washes after | 1-3 | |
| incubation) | | |
| Post wash force (centrifugal force in washing step | 300-600 | g |
| after incubation) | | |
| Post wash time (centrifugation time in washing step | 300-900 | s |
| after incubation) | | |
| Final volume (volume of adjusted system after | 10-100 | ml |
| incubation) | | |

In one embodiment, in step (b), the sorting comprises positive sorting and/or negative sorting.

In one embodiment, in step (b), the sorting comprises sorting by sorting magnetic beads which contain binding agents specific to at least one immune cell surface marker (such as CD4 and/or CD8). The sorting magnetic beads bind to the immune cell surface marker(s) through the binding agents to form a sorting magnetic bead-cell complex, so as to obtain a first immune cell population enriched in immune cells.

In one embodiment, the binding agents are antibodies. In one embodiment, the antibodies are specific antibodies. In one embodiment, the antibodies are anti-CD4 antibodies, anti-CD8 antibodies, or a combination thereof.

In one embodiment, the cell surface markers are: CD4, CD8, or a combination thereof.

In one embodiment, the binding agents specifically bind to the cell surface marker(s).

In one embodiment, the sorting magnetic beads contain anti-CD4 antibodies and/or anti-CD8 antibodies.

The step of sorting/separating immune cells may comprise one, two or more, or a combination of, positive enrichment steps, i.e., separation of T cells, T cell subsets and/or T cell progenitors (direct magnetic labeling). T cells may be selected for CD4+ and/or CD8+ T cells by using antigen binding molecules coupled to particles such as magnetic beads specific for CD4 and/or CD8. A subpopulation of T cells such as naïve and central memory T cells may be separated e.g., by using the marker CD62L.

The step of sorting/separating immune cells may also comprise negative enrichment (direct labeling of non-immune cells such as non-T cells) of immune cells such as T cells, or of the depletion of cellular subsets to be removed from the preparation. For example, B cells may be removed via the CD19 marker. Inhibitory cells such as regulatory T cells (CD25 high), monocyte (CD14) can be removed as well using the markers CD25 and CD14, respectively.

In one embodiment, the washing liquid is a buffer.

In one embodiment, the sorting liquid is a buffer containing sorting magnetic beads.

In one embodiment, the buffer is a pH 6.8-7.4 phosphate-buffered saline (PBS) buffer.

In one embodiment, in step (c), the activating magnetic beads are activating magnetic beads specific to CD3, activating magnetic beads specific to CD28, or a combination thereof.

In one embodiment, the activating magnetic beads are Dynabeads®.

In one embodiment, in step (c), the density of the first (or second) immune cell population ranges from about 0.5×10⁶ cells/ml to about 10×10⁶ cells/ml.

In one embodiment, in step (e), the viruses are lentiviruses.

In one embodiment, the gene is a tumor-killing gene.

In one embodiment, in step (e), in the transduction/transfection step, the ratio of the number of viruses to the number of cells ranges from about 1:1 to about 10:1.

In one embodiment, in step (f), the culturing is carried out in Wave equipment.

In one embodiment, the culturing is carried out in a wave culture bag.

In one embodiment, the wave culture bag has a capacity of 2 L-10 L.

In one embodiment, Wave parameters are as follows: temperature of 35-39° C., gas flow of 0.08-0.15 L/min, 4-6% of CO₂, rocking speed of 10-18 rpm, and rocking angle of 6-10°.

In one embodiment, in step (f), after culturing, the activating beads are removed using a magnetic device (e.g., CTS™ DynaMag™ Magnet).

In one embodiment, step (f) further comprises concentrating the cultured fourth immune cell population. The concentrating may be carried out on the Sepax C-pro equipment.

In one embodiment, a “CultureWash” program of Sepax C-pro is used for concentration. The “CultureWash” parameters may comprise one or more of the following:

| Parameter | Set value |
|---|---|
| Initial volume (volume of the cell mixture | 10 to 1200 ml |
| after removing the activating beads) | |
| Final Volume (concentrated volume) | 5-500 ml |
| Intermediate Volume (volume of the | 5 to 50 ml |
| precipitate after centrifugation) | |
| G-force (centrifugal force in washing and | 300 to 400 g |
| concentration steps) | |
| Sedimentation time (centrifugal force in | 300 to 600 s |
| washing step) | |
| Wash Cycles (number of washes) | 1 to 3 |

In another aspect, the present disclosure provides a substantially pure composition of genetically modified immune cells. In certain embodiments, the present method generates about 80% to about 100%, at least or about 50%, at least or about 55%, at least or about 60%, at least or about 65%, at least or about 70%, at least or about 75%, at least or about 80%, at least or about 85%, at least or about 90%, at least or about 91%, at least or about 92%, at least or about 93%, at least or about 94%, at least or about 95%, at least or about 96%, at least or about 97%, at least or about 98%, at least or about 99%, or about 100% of the desired immune cells (e.g., genetically modified immune cells) in the cell composition or pharmaceutical composition.

In one embodiment, a sample comprising immune cells is centrifuged (such as using optical density phase detection). Excess erythrocytes are removed. The cells are washed to avoid cell aggregation, and magnetically labeled with a magnetic cell separation reagent. After labeling, cells are washed, magnetically enriched via an integrated magnetic cell selection column and then returned to a cell culture chamber.

The immune cells may be activated with one or a combination of activating agents capable of inducing immune cell (e.g., T cell) proliferation, such as agonistic antibodies (e.g., anti-CD3 and anti-CD28), cytokines (e.g. IL-1b, IL-2, IL-4, IL-6, IL-7, IL-9, IL-10, IL-12, IL-15, IL-17, IL-21, IL-22, IL-23, IL-35, TGF-b, IFN alpha, IFN gamma, TNF alpha), recombinant proteins, costimulatory molecules, lectins, ionophores, synthetic molecules, antigen presenting cells (APCs), artificial APCs, feeders, and combinations thereof.

In one embodiment, after a period of culturing the immune cells, viral vectors are added, and the cells are transduced. Following a further cell culture period, the cells can be transduced again or washed and harvested (formulated). Prior to in vivo transfer of the genetically modified immune cell products, the cells may be washed, concentrated and resuspended in a buffer compliant with clinical requirements for in vivo infusion.

In one embodiment, immune cells are labeled by binding to antibody-coupled magnetic beads to a cell surface marker present on the surface of the immune cells, and the labeled cells are enriched by magnetic separation (positive enrichment).

In another embodiment, the immune cells are enriched by binding to antibody-coupled magnetic beads to a cell surface marker not present on the surface of the immune cells such as T cells or defined cellular subsets, and depleting the labeled cells by magnetic separation (negative enrichment).

In a further embodiment, in addition to the first enrichment of immune cells, the genetically modified immune cells are enriched in a second enrichment step by magnetic labeling of the genetically modified immune cells, and magnetic separation before or after cultivation to obtain higher percentage/amount of the genetically modified immune cells in the final cell composition obtained by the present method. For example, if the genetically modified cell is a T cell expressing a CAR or TCR, the second separation step may be performed by using an antigen-binding molecule coupled to a magnetic particle specific for the recombinantly expressed CAR or TCR on the cell surface of the genetically modified T cell.

In one embodiment, a sample, e.g., whole blood from a patient, comprising immune cells, is provided. The cell sample may be centrifuged to separate erythrocytes and platelets from other cells including immune cells. Magnetic separation of immune cells may be performed by using antibodies coupled to magnetic particles specific for markers of immune cells, such as CD2, CD3, CD4, CD8, CD25, CD28, CD27, CD45RA, CD45RO, CD62L, CD95, CD127, CD137, alpha/beta TCR, gamma/delta TCR, CCR7, PD-1, Lag3, and combinations thereof. Passing the labeled cells through magnetic device (e.g., a magnet unit with separation column) results in an enrichment of the immune cells. The separated immune cells may be set at a given density (e.g., 0.5×10⁶ cells per ml to 2×10⁶ cells per ml) to be activated by using activating agents described herein. The activated immune cells are then genetically modified, e.g., they are transduced with a lentiviral vector comprising a polynucleotide sequence encoding a CAR. Then the genetically modified immune cells may be expanded. Finally, the cultured cells may be washed by centrifugation, thereby allowing the replacement of culture medium with a buffer appropriate for subsequent applications such as infusion of the generated cell composition to a patient.

In one embodiment of the invention, a higher purity of transduced immune cells, e.g., T cells expressing a transgene such as a CAR or TCR on their cell surface, is obtained at the end of the manufacturing process. An additional cell selection step may be used to specifically enrich the genetically modified immune cells. For example, magnetic particles coated with antibodies directed against the surface molecule encoded by the transgene may be used for the selection step. The step of enrichment may be carried out by using a magnetic separation unit and may be done before final formulation.

In certain embodiments, a selection agent that can be removed from the surface of the selected cells after this second enrichment and before application to a patient or downstream use is used.

In certain embodiments, the immune cells may be activated in suspension. Immune cells can be further modified using lentiviral vector and expanded in suspension. Shaking conditions may be maintained during the activation, genetic modification and expansion steps of the process as disclosed herein to keep the cells in suspension.

In one embodiment, the immune cells may be genetically modified using lentiviral vectors. In one embodiment, lentiviral vectors with the VSVG pseudotype enable efficient transduction. Other types of lentiviral vectors may also be used, such as measles virus (ML-LV), gibbon ape leukaemia virus (GALV), feline endogenous retrovirus (RD114), baboon endogenous retrovirus (BaEV) derived pseudotyped envelopes). Other viral vectors such as gamma or alpha retroviral vectors can be used. Transduction enhancer reagents may be added.

In a further aspect, the present disclosure provides a pharmaceutical composition comprising the genetically modified immune cells.

The sample may be a human cell sample of hematologic origin. For example, the cell sample may be composed of hematologic cells from a donor or a patient. Such blood product can be in the form of whole blood, buffy coat, leukapheresis, PBMCs or any clinical sampling of blood product. It may be from fresh or frozen origin. Samples include, but are not limited to, fresh peripheral blood, fresh apheresis collections, cryopreserved apheresis collections, PBMC collections, T cells, NK cells, etc.

The centrifugation step may comprise one, more or all of the following aspects: gradient separation, erythrocyte reduction, platelet removal and cell washing.

In certain embodiments, washing means the replacement of the medium or buffer in which the cells are kept. The replacement of the supernatant can be in part or entirely. Several washing steps may be combined in order to obtain a more complete replacement of the original medium in which the cells are kept. A washing step may involve pelleting the cells by centrifugation forces and removing the supernatant.

The term “marker” may refer to a cell antigen that is specifically expressed by a certain cell type. Preferentially, the marker is a cell surface marker so that enrichment, isolation and/or detection of living cells can be performed. The markers may be positive selection markers such as CD4, CD8 and/or CD62L, or may be negative selection markers (e.g., depletion of cells expressing CD14, CD16, CD19, CD25, CD56).

The term “antigen-binding molecule” as used herein refers to any molecule that binds preferably to, or is specific to, the desired target molecule, i.e., the antigen. The term “antigen-binding molecule” comprises, e.g., an antibody or antibody fragment. The term “antibody” as used herein may refer to polyclonal or monoclonal antibodies. The antibody may be of any species, e.g., murine, rat, sheep, human, etc. For therapeutic purposes, if non-human antigen binding fragments are to be used, these can be humanized by any method known in the art. The antibodies may also be modified antibodies (e.g., oligomers, reduced, oxidized and labeled antibodies). The term “antibody” comprises both intact molecules and antibody fragments, such as Fab, Fab′, F(ab′)2, Fv and single-chain antibodies. Additionally, the term “antigen-binding molecule” includes any molecule other than antibodies or antibody fragments that binds preferentially to the desired target molecule of the cell. Suitable molecules include, without limitation, oligonucleotides known as aptamers that bind to desired target molecules, carbohydrates, lectins or any other antigen binding protein (e.g., receptor-ligand interaction).

The linkage (coupling) between the antigen-binding molecule (e.g., antibody or antibody fragment) and beads/particles can be covalent or non-covalent. A covalent linkage may be, e.g., the linkage to carboxyl-groups on polystyrene beads, or to NH2 or SH2 groups on modified beads. A non-covalent linkage may be, e.g., via biotin-avidin or a fluorophore-coupled-particle linked to anti-fluorophore antibody.

A potent sorting technology is magnetic cell sorting. Methods to separate cells magnetically are commercially available e.g., from Invitrogen, Stem cell Technologies, in Cellpro, Seattle or Advanced Magnetics, Boston. For example, monoclonal antibodies can be directly coupled to magnetic polystyrene particles such as Dynabeads® or similar magnetic particles and used, e.g., for cell separation. The cells are isolated, e.g., by placing the tube on a magnetic rack. These microbeads can be either directly conjugated to monoclonal antibodies or used in combination with anti-immunoglobulin, avidin or anti-hapten-specific microbeads. Cells can be separated by incubating them with magnetic microbeads coated with antibodies directed against one or more particular surface antigens. This causes the cells expressing this antigen to attach to the magnetic microbeads. With this method, the cells can be separated positively or negatively with respect to the particular antigen(s). The procedure can be performed using direct magnetic labeling or indirect magnetic labeling. For direct labeling, the specific antibody is directly coupled to the magnetic microbeads. In indirect labeling, a primary antibody, a specific monoclonal or polyclonal antibody, a combination of primary antibodies, directed against any cell surface marker can be used. The primary antibody can either be unconjugated, biotinylated, or fluorophore-conjugated. The magnetic labeling is then achieved with anti-immunoglobulin microbeads, anti-biotin microbeads, or anti-fluorophore microbeads.

The term “genetically modified cell” means cells containing and/or expressing a transgene (or foreign gene) or nucleic acid sequence which in turn modifies the genotype or phenotype of the cell or its progeny. The term may refer to the fact that cells can be manipulated by recombinant methods well known in the art to express stably or transiently peptides or proteins, e.g., CARs, which are not expressed in these cells in the natural state.

Genetic modification of cells may include, but is not limited to, transfection, electroporation, nucleofection, transduction using retroviral vectors, lentiviral vectors, non-integrating retro- or lentiviral vectors, transposons, designer nucleases including zinc finger nucleases, TALENs or CRISPR/Cas.

The genetically modified immune cells, obtainable by the methods disclosed herein, may be used for subsequent steps such as research, diagnostics, pharmacological or clinical applications known to the person skilled in the art.

The genetically modified immune cells can also be used as a pharmaceutical composition in therapy, e.g., cellular therapy, or prevention of diseases. The pharmaceutical composition may be transplanted into an animal or human, for example a human patient. The pharmaceutical composition can be used for the treatment and/or prevention of diseases in mammals, especially humans, possibly including administration of a pharmaceutically effective amount of the pharmaceutical composition to the mammal. Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The composition of the genetically modified immune cells, obtained by the present method, may be administered either alone, or as a pharmaceutical composition in combination with diluents and/or with other components such as cytokines or cell populations. Briefly, the present pharmaceutical composition may comprise the genetically modified immune cells, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives.

Cell sorting includes, but is not limited to, positive sorting based on, e.g., CD4⁺, CD8⁺, CD62L⁺, CD3⁺, CD56⁺ and combinations thereof, and negative sorting based on, e.g., CD14⁺, CD19⁺, CD269⁺ and combinations thereof.

Cell activation density may range from 0.1×10⁶/ml to 20×10⁶/ml.

In certain embodiments, the microbeads (e.g., Dynabeads®) are monodisperse/homogeneous, superparamagnetic and polymeric microspheres comprising γFe₂O₃ and Fe₃O₄ magnetic materials. The microbeads are coated with a layer of polymeric material, which acts as a carrier for adsorbing or binding antibodies specific for CD3 and/or CD28 cell surface molecules.

Main advantages of the present method include the following. The present cell preparation method can quickly prepare immune cells, reduce the costs, improve production capacity, and is suitable for industrial production. The immune cells prepared by the present method have high quality and can ensure clinical efficacy.

Vectors derived from retroviruses such as lentiviruses may be used to achieve long-term gene transfer because they allow long-term, stable integration of transgenes and propagation of the transgenes in daughter cells.

The nucleic acid can be cloned into many types of vectors. For example, the nucleic acid can be cloned into such vectors, which include, but are not limited to, plasmids, phagemids, phage derivatives, animal viruses, and cosmids. Specific vectors of interest include expression vectors, replication vectors, probe generation vectors, and sequencing vectors.

Further, the expression vectors can be provided to cells in the form of viral vectors. Viruses that can be used as vectors include, but are not limited to, retroviruses, adenoviruses, adeno-associated viruses, herpes viruses, and lentiviruses.

Methods for introducing genes into cells and expressing genes into cells are known in the art. For example, the expression vector can be transferred into the host cell by physical, chemical or biological means.

The physical methods for introducing polynucleotides into host cells include calcium phosphate precipitation, lipofection, particle bombardment, microinjection, electroporation, etc. Methods for producing cells that comprise vectors and/or exogenous nucleic acids are well known in the art. A method for introducing polynucleotides into host cells is calcium phosphate transfection.

Biological methods for introducing polynucleotides of interest into host cells include the use of DNA and RNA vectors. Viral vectors, especially retroviral vectors, have become the most widely used methods for inserting genes into mammalian cells such as human cells. Other viral vectors can be derived from lentiviruses, poxviruses, herpes simplex viruses I, adenoviruses, adeno-associated viruses, etc.

In certain embodiments, the vector is a lentiviral vector.

Chemical means for introducing polynucleotides into host cells include colloidal dispersion systems, such as macromolecular complexes, nanocapsules, microspheres, and beads; and lipid-based systems, including oil-in-water emulsions, micelles, mixed micelles, and liposomes. Exemplary colloidal systems used as delivery vehicles in vitro and in vivo are liposomes (e.g., artificial membrane vesicles).

In the case of using a non-viral delivery system, an exemplary delivery tool is a liposome. The use of a lipid preparation is considered to introduce nucleic acids into host cells (in vitro, ex vivo or in vivo). On the other hand, the nucleic acids can be associated with lipids. Lipid-associated nucleic acids can be encapsulated in the aqueous interior of liposomes, dispersed in the lipid bilayer of liposomes, and attached to liposomes via linking molecules associated with both liposomes and oligonucleotides, trapped in liposomes, complexed with liposomes, dispersed in a solution containing lipids, mixed with lipids, combined with lipids, contained in lipids as a suspension, contained in micelles or complexed with micelles, or associated with lipids by other methods. The lipids, lipids/DNA or lipids/expression vectors associated with the composition are not limited to any specific structure in the solution. For example, they can exist in a bilayer structure, as micelles or have a “collapsed” structure. They can also simply be dispersed in a solution, possibly forming aggregates in uneven sizes or shapes. Lipids are fatty substances and can occur naturally or be synthesized. For example, lipids include fat droplets, which naturally occur in cytoplasm and in compounds containing long-chain aliphatic hydrocarbons and their derivatives such as fatty acids, alcohols, amines, amino alcohols and aldehydes.

The present invention will be further explained below in conjunction with specific embodiments. It should be understood that these embodiments are only used to illustrate the present invention and not to limit the scope of the present invention. The experimental methods that do not indicate specific conditions in the following embodiments usually follow the conventional conditions or the conditions recommended by the manufacturer. Unless otherwise stated, the percentages and parts are calculated by weight.

### Example 1 Preparing Cells for Cellular Immunotherapy

### 1. Cell Culture:

All cells were cultured under normal cell culture conditions.

### 2. Preparation Method

### (1) Providing Cell Samples

10 to 440 ml of a cryopreserved sample (a blood sample) containing CD4⁺ and CD8⁺ cells was thawed as cell samples.

### (2) Washing and Sorting

A Sepax C-Pro “BeadWash” program was used to wash the cells which were then incubated with sorting magnetic beads containing anti-CD4 antibodies and anti-CD8 antibodies. The parameters are shown in Table 1.

TABLE 1
Sepax Pro Beadwash parameter settings

| Parameter | Set value | |
|---|---|---|
| Initial Volume (volume of the samples) | 10-440 | ml |
| Dilution ratio (volume ratio of washing liquid to | 1-2 | |
| liquid to be washed) | | |
| Dilution speed (speed of adding washing liquid) | 17-60 | ml/min |
| Intermediate volume (volume of precipitate after | 5 to 20 ml | |
| centrifugation) | | |
| Pre-wash cycle (number of washes before | 1 | |
| incubation step) | | |
| Pre-wash force (centrifugal force in washing step) | 300 to 600 g | |
| Pre-wash time (centrifugation time in washing step) | 300 to 600 s | |
| Reagent volume | 15-20 | ml |
| Incubation volume | Calculated | |
| | incubation volume | |
| | of magnetic beads | |
| Incubation time (time for incubation) | 30 | min |
| Post wash cycle (number of washes after | 1 | |
| incubation) | | |
| Post wash force (centrifugal force in washing step | 300 | g |
| after incubation) | | |
| Post wash time (centrifugation time in washing step | 900 | s |
| after incubation) | | |
| Final volume (volume of adjusted system after | 100 | ml |
| incubation) | | |

The Sepax Pro “BeadWash” program was as follows.

A washing liquid of pH 7.2 PBS buffer was added to the cell sample, mixed and centrifuged. The supernatant was removed to obtain a precipitate.

A sorting solution was added to the precipitate and incubated for 10-30 min to obtain an incubation mixture, where the sorting solution was the pH 7.2 PBS buffer containing the magnetic beads. The volume of the sorting magnetic beads=CD4/CD8 lymphocyte volume/[(200−800)×10⁶/ml]. The sorting magnetic beads contained anti-CD4 antibodies and anti-CD8 antibodies which can specifically bind to the CD4 and CD8 cell surface markers to form sorting magnetic bead-cell complexes.

In step (2), if the incubation time was too short, the binding of the target cells would be affected. Specifically, some of the target cells cannot bind to the sorting magnetic beads, which affected the sorting efficiency. When the incubation time was too long, the growth of the cells was not optimal. When the volume of the sorting magnetic beads was too low, some of the target cells cannot be labeled, which affected the sorting efficiency. When the amount of the magnetic beads was too high, it will increase the amount of free magnetic beads after incubation and washing, occupying the binding sites of the sorting column, affecting the sorting efficiency.

CliniMacs equipment was used to separate the sorting magnetic bead-cell complexes in pH 7.2 PBS buffer from the incubation mixture.

The CliniMacs equipment worked as follows.

First, a magnetic field was used to retain the sorting magnetic bead-cell complexes while liquid in the incubation mixture was removed. Then, the magnetic field was removed, and the sorting magnetic bead-cell complexes were rinsed with a pH 7.2 PBS buffer to obtain sorting magnetic beads-cell complexes in pH 7.2 PBS buffer.

### (3) Cell Activation

The sorting magnetic bead-cell complexes in pH 7.2 PBS buffer were centrifuged. The supernatant was removed to obtain a precipitate containing the sorting magnetic bead-cell complexes. After re-suspension in a culture medium, a cell culture solution containing activating magnetic beads (Dynabeads®) for activating the cells was added to obtain a cell mixture. Subsequent incubation was carried out for 12-24 hours. In the cell mixture, the ratio of the number of activating magnetic beads to the number of cells was 0.5-5:1, and the density of the activated cells was 0.5-10×10⁶ cells/ml.

In step (4), if the amount of the activating magnetic beads was too high, excessive activation of the lymphatic T cells may occur and the residual amount may increase when the magnetic beads were removed. Excessive activation of the cells may result in cell apoptosis and differentiation. Excessive amounts of the magnetic beads can easily overload the allowed capacity, resulting in excessive residual magnetic beads in the final cell product. When the activation density was too high or too low, it may affect the binding of the cells and the activating magnetic beads, thereby affecting the activation efficiency of the magnetic beads on the cells, and then affecting the expansion of the cells.

### (4) Transduction

After culturing the cell mixture for 2 days, the cells were transduced with viruses carrying a target gene, with the multiplicity of infection (MOI) of the viruses to the cells being 1-10:1. After incubating for 2 days, an incubation mixture was obtained. The same volume of cell culture medium was added to the incubation mixture to dilute it by 1-fold. After culturing for 1 additional day, the cells were inoculated into Xuri Wave and cultured for 1-2 days. The cell culture parameters were as follows: temperature of 37° C., gas flow of 0.1 L/min, 5% of CO₂, rocking speed of 10-18 rpm, and rocking angle of 6-10°. Then a cell mixture was obtained.

In step (4), too low or too high culture temperature in Xuri Wave may affect the metabolic growth rate of the cells. Too low or too high CO₂ ratio, gas flow rate, and/or rocking angle and speed may affect the oxygen dissolving rate and other culture conditions, thus inhibiting cell growth. The above parameters can be controlled using Wave culture.

### (5) Removing the Activating Magnetic Beads

CTS™ Dynamag™ equipment was used to remove the activating magnetic beads from the cell mixture carrying the target gene.

(6) Cell Culture with Perfusion

After the activating magnetic beads removal, the cells were cultured with perfusion (the culture medium was replenished, and the cell culture medium was kept at 500 ml) to obtain a cultured cell mixture. The perfusion parameters are shown in Table 2.

TABLE 2
Cell perfusion culture

| Perfusion rate | Cell density |
|---|---|
| No perfusion | Cell density < 2 × 10⁶ cell/ml |
| 0.25 to 0.5 L/day | 2 × 10⁶ cell/ml ≤ cell density < 4 × 10⁶ cell/ml |
| 0.5 to 1 L/day | 4 × 10⁶ cell/ml ≤ cell density |

### (7) Cell Concentration, Aliquoting and Lyophilization

After washing and concentration of the cells obtained in step (6), a cryopreservation composition was added. Aliquoting and lyophilization were carried out to obtain genetically modified cells.

A “CultureWash” program of Sepax C-pro was used for the cell washing and concentration steps. The CultureWash parameters are shown in Table 3.

TABLE 3
CultureWash parameter settings

| Parameter | Set value |
|---|---|
| Initial volume (volume of the cell mixture | 10 to 1200 ml |
| after removal of activating magnetic beads) | |
| Final Volume (concentrated volume) | 5-500 ml |
| Intermediate Volume (volume of | 5 to 50 ml |
| precipitate after centrifugation) | |
| G-force (centrifugal force in washing and | 300 to 400 g |
| concentration steps) | |
| Sedimentation time (centrifugal force in | 300 to 600 s |
| washing step) | |
| Wash Cycles (number of washes) | 1 to 3 |

### 3. Experimental Results

According to the above method, parallel experiments were carried out a number of times to investigate different process parameters, and the results are as follows.

3.1. In the washing and incubation of step (2), a Sepax Pro “BeadWash” program was used to carry out washing and incubation steps. The recovery rates of monocytes and lymphocytes are shown in Table 4.

TABLE 4
Recovery rate of monocytes and lymphocytes

| | Monocytes | Lymphocytes |
|---|---|---|
| | (number) | (number) |
| Washing and incubation | 2052.0 × 10⁶ | 1512.0 × 10⁶ |
| “BeadWash” washing | 2022.2 × 10⁶ | 1391.4 × 10⁶ |
| and incubation | | |
| Recovery rate | 98.5% | 92.0% |

Table 4 shows that after the washing and incubation of the cell sample with the Sepax Pro “BeadWash” program, the cell recovery rate was greater than 90%, showing little cell loss.

3.2 In the sorting step of step (3), CliniMacs was used for cell sorting. The recovery rate of cells is shown in Table 5.

TABLE 5
Recovery rate of cells

| Number of CD4⁺CD8⁺T cells in the cell sample | 1832.7 × 10⁶ |
|---|---|
| Number of CD4⁺CD8⁺T cells after sorting | 1500 × 10⁶ |
| Recovery rate of CD4⁺CD8⁺T cells after sorting | 82% |

Table 5 shows that the recovery rate of using CliniMacs to carry out cell sorting was greater than 80%, showing that CliniMacs can collect most of the target cells.

3.3 In the transduction step of step (5), the cell activation ratio was assayed on the first day when the cells were cultured. The cells were incubated for 2 days after viral transduction. Table 6 shows the cell activation ratio on the first day, the cell number and positive rate of cells on the third day and the fourth day.

TABLE 6
Cell activation of step (4)

| | |
|---|---|
| Cell number in cell mixture | 656.11 × 10⁶ |
| Cell activation rate after culturing for 1 day | 85% |
| Cell number 1 day post virus transduction | 1349.94 × 10⁶ |
| Positive rate 1 day post virus transduction (i.e., | 46.10% |
| percentage of cells expressing the transgene) | |
| Cell number 2 days post virus transduction | 2360.0 × 10⁶ |
| Positive rate 2 days post virus transduction (i.e., | 56.80% |
| percentage of cells expressing the transgene) | |

Table 6 shows that in step (4) after the cells were cultured for 1 day, about 85% of the cells were activated, showing excellent cell activation efficiency. Culturing the cells for additional time was accompanied by cell expansion and increased transgene expression.

3.4 In the cell preparation method, the cell subpopulation properties of the sample (the blood sample) in step (1), after the sorting of step (3), and when continuing to culture the cells for 2 days after the transduction step in step (5) are shown in Table 7.

TABLE 7
Cell subpopulation properties

| | Tnaive (classification) | Tcm |
|---|---|---|
| Cell sample | 12.59% | 25.81% |
| After sorting | 12.59% | 20.78% |
| 2 days post virus | 36.12% | 61.80% |
| transduction | | |

Tnaive: naive T cell; Tcm: central memory T cell.

Table 7 shows that during the cell preparation process, the ratio of Tnaive to Tcm cells continuously increased, showing that the cell activity continuously enhanced during the preparation process.

3.5 The cell recovery rate and viability before and after the use of CTS™ Dynamag™ for removing Dynabeads® are shown in Table 8.

TABLE 8
Cell recovery rate and viability before and after
removal of Dynabeads ® by CTS ™ Dynamag ™

| | Cell number | Cell viability |
|---|---|---|
| Before removal | 1087.9 × 10⁶ | 96.2% |
| After removal | 933.26 × 10⁶ | 94.2% |
| Recovery rate | 85.8% × 10⁶ | N/A |

Table 8 shows that the removal of Dynabeads® by CTS™ Dynamag™ provided a higher recovery rate and viability of the cells.

3.6 Cell Washing and Concentration

The cell recovery and survival rates before and after washing and concentration of the sample with Sepax Pro are shown in Table 9.

TABLE 9
Cell recovery and survival rates before
and after washing and concentration

| | Cell number | Cell viability |
|---|---|---|
| Before washing and concentration | 541.68 × 10⁶ | 94.20% |
| After washing and concentration | 480.20 × 10⁶ | 92.15% |
| Recovery rate | 88.7% | N/A |
| | Set value/ | Actual |
| | theoretical value | volume |
| Volume of cell suspension after | 30 ml | 30.1 ml |
| concentration | | |

Table 9 shows that washing and concentration using Sepax Pro offered excellent cell recovery and survival rates.

3.7 Cell Aliquoting

Cell Connect aliquoting tubing or Sefia was used to aliquot the cells, which can ensure the accuracy of the output volume and the consistency of the cell density during aliquoting. The results are shown in Table 10.

TABLE 10

| | Set value/ | Actual |
|---|---|---|
| | Theoretical value | volume |
| Cell Connect aliquoting tubing | | |
| Sample volume of aliquoted | 88 ml | 86 ml |
| first bag | | |
| Sample concentration of | 16 × 10⁶/ml | 16.4 × 10⁶/ml |
| aliquoted first bag | | |
| Sample volume of aliquoted | 880 ml | 86 ml |
| second bag | | |
| Sample concentration of | 16 × 10⁶/ml | 16.4 × 10⁶/ml |
| aliquoted second bag | | |
| Sefia | | |
| Sample volume of aliquoted | 88 ml | 86.2 ml |
| first bag | | |
| Sample concentration of | 12 × 10⁶/ml | 12 × 10⁶/ml |
| aliquoted first bag | | |
| Sample volume of aliquoted | 88 ml | 82.4 ml |
| second bag | | |
| Sample concentration of | 12 × 10⁶/ml | 11.7 × 10⁶/ml |
| aliquoted second bag | | |

The scope of the present disclosure is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present disclosure and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present disclosure have been shown and described, it will be obvious to those skilled in the art, that changes and modifications may be made without departing from the spirit and scope of the invention. The matter set forth in the foregoing description and accompanying drawings is offered by way of illustration only and not as a limitation.

1. A method for preparing genetically modified immune cells, the method comprising:
(a) providing a sample containing immune cells;
(b) sorting the sample to obtain a first immune cell population enriched in immune cells;
(c) activating the first immune cell population to obtain a second immune cell population;
(d) culturing the second immune cell population to obtain a third immune cell population;
(e) genetically modifying the third immune cell population to obtain a fourth immune cell population; and
(f) culturing the fourth immune cell population to obtain genetically modified immune cells.

2. The method of claim 1, wherein in step (c) the first immune cell population is activated with microbeads coated with activating agents.

3. The method of claim 2, wherein the activating is performed with a microbead-to-cell ratio ranging from about 0.5:1 to about 5:1.

4. The method of claim 2, wherein the activating agents are selected from the group consisting of: antibodies or fragments thereof, cytokines, recombinant costimulatory molecules, small drug inhibitors, and combinations thereof.

5. The method of claim 1, wherein the activating agents are anti-CD3 and/or anti-CD28 antibodies or fragments thereof.

6. The method of claim 1, wherein in step (c) the activating is performed with a density of the first immune cell population ranging from about 0.5×10⁶ cells/ml to about 10×10⁶ cells/ml.

7. The method of claim 1, wherein in step (b) the sorting is performed by mixing the sample with sorting magnetic beads.

8. The method of claim 7, wherein the sorting comprises positive sorting and/or negative sorting.

9. The method of claim 7, wherein the positive sorting comprises using anti-CD4 and/or anti-CD8 antibodies or fragments thereof.

10. The method of claim 1, wherein in step (f), for the culturing, when the fourth immune cell population has a density of less than 2×10⁶ cells/ml, no perfusion is carried out; when the fourth immune cell population has a density of greater than or equal to 2×10⁶ cells/ml and less than 4×10⁶ cells/ml, perfusion is carried out at a rate of 0.5 V/day to 1 V/day; and when the fourth immune cell population has a density of greater than or equal to 4×10⁶ cells/ml, perfusion is carried out at a rate of 1 V/day to 2 V/day, wherein V is the volume of a culture system.

11. The method of claim 1, wherein step (b) to step (f) are performed in about 4 days to about 5 days.

12. The method of claim 1, wherein step (d) is performed for about 1.5 days to about 3 days.

13. The method of claim 1, wherein step (e) is performed for about 0.5 days to about 2.5 days.

14. The method of claim 1, wherein step (f) is performed for about 1 day to about 3.5 days.

15. The method of claim 1, wherein the immune cells are T cells or T cell subsets.

16. The method of claim 1, wherein in step (e) the genetically modifying is transducing or transfecting.

17. The method of claim 1, wherein in step (e) the genetically modifying comprises introducing into the third immune cell population a polynucleotide encoding a chimeric antigen receptor (CAR) or a T cell receptor (TCR).

18. The method of claim 1, wherein in step (e) the genetically modifying comprises transducing the third immune cell population with lentiviral vectors, gamma-retroviral vectors, alpha-retroviral vectors, or adenoviral vectors.

19. (canceled)

20. The method of claim 1, wherein the sample is peripheral blood, cells, fresh apheresis, cryopreserved apheresis, monocyte collections, peripheral blood mononuclear cells (PBMCs), or combinations thereof.

21. (canceled)

22. Genetically modified immune cells prepared by the method of claim 1.

23-24. (canceled)
